(19) United States
(12) Patent Application Publication
(10) Pub. No.: US 2021/0282395 A1
(43) Pub. Date: Sep. 16, 2021

(54) TOPICAL ANTISEPTIC SKIN SANITIZING NITROGEN SPRAY

(71) Applicant: Alim Enterprises, LLC, Santa Monica, CA (US)

(72) Inventors: Ross Sklar, Santa Monica, CA (US); William Arnold Williams, Cornelius, NC (US)

(21) Appl. No.: 16/869,289

(22) Filed: May 7, 2020

Related U.S. Application Data
(60) Provisional application No. 62/988,875, filed on Mar. 12, 2020.
(60) Provisional application No. 63/009,445, filed on Apr. 13, 2020.

(51) Int. Cl. A01N 31/02; A61K 31/045; A61K 9/00

(52) U.S. Cl. CPC A01N 31/02; A61K 9/0014; A61K 31/045

(57) ABSTRACT

A method for topical disinfection of mammalian skin comprising the steps of: (a) filling a container body with a lower alcohol selected from the group of C₁-C₆ alcohols or a mixture thereof at a concentration of from 24.00% to 45.00% vol/vol, water at a concentration of from 8.00% to 12.00% vol/vol, and a hydrophilic humectant at a concentration of from 0.04% to 4.25% vol/vol; (b) attaching and sealing a valve assembly to the container body; (c) filling the container body sealed with a valve assembly under a pressure of from 80 psi to 120 psi with nitrogen, (d) attaching an actuator to the valve assembly; and (e) depressing the actuator and dispensing onto mammalian skin. 15 seconds after dispensing, the method achieves at least a 3−log₁₀ (99.9%) reduction in the concentration Gram-negative and Gram-positive microorganisms in accordance with method ASTM E2315-03(2008).

### CROSS-REFERENCE TO RELATED APPLICATIONS

The present application claims the benefit of the following U.S. patent applications, the entire contents of which are hereby incorporated by reference: U.S. Provisional Patent Application Ser. No. 62/988,875 (filed on Mar. 12, 2020); U.S. Provisional Patent Application Ser. No. 63/007,427 (filed on Apr. 9, 2020), U.S. Provisional Patent Application Ser. No. 63/009,445 (filed on Apr. 13, 2020).

### FIELD OF INVENTION

Methods for rapid topical disinfection of mammalian skin and statistically significant (>3−log₁₀ CFU/ml) reduction of microorganisms thereon, including Gram-positive and Gram-negative bacteria, and envelope viruses, including coronavirus.

### BACKGROUND OF THE INVENTION

In April 2019, the US Food and Drug Administration (the FDA) issued a final action with respect to the use of certain active ingredients in nonprescription [also known in the art as over-the-counter (OTC)] consumer antiseptic rub products. The FDA action is based on the Agency's 2016 proposed OTC Consumer Antiseptic Rub rule, which, in turn, is based on a Tentative Final Monograph (TFM) issued in 1994. The FDA agreed to defer its decision on inclusion of ethanol and isopropanol to allow more time for interested parties to complete studies and submit additional safety and efficacy data. During this extended submission period, the TFM applies to the marketing of alcohol-based hand sanitizers. According to the 1994 TFM, ethanol should be used in a hand sanitizer at a concentration between 60-95% (v/v) in an aqueous solution; and isopropyl alcohol should be used at a concentration between 70-91.3% (v/v). See, US FDA, “Temporary Policy for Manufacture of Alcohol for Incorporation Into Alcohol-Based Hand Sanitizer Products During the Public Health Emergency (COVID-19)”, (issued Mar. 20, 2020; updated Mar. 27, 2020).

In approximately 2014, Veltek Associates, Inc. (Malvern, Pa.) began selling a bag-on-valve continuous spray sterile isopropanol solution for use as a hard surface cleanroom disinfectant under the brand name DECON-AHOL®. The DECON-AHOL® disinfectant is subject to regulation and approval by the Environmental Protection Agency (EPA), not the FDA. DECON-AHOL® is not intended or used for direct human contact. The EPA-approved label for DECON-AHOL® includes directions that if skin contact occurs, “rinse skin immediately for 10-15 minutes”; and warns that “prolonged or frequently repeated skin contact may cause allergic reactions.”

European Patent EP0848907B1 (issued in German) relates to an aqueous-alcoholic spray disinfectant preparation for inactivating coated and non-coated viruses on inanimate surfaces. See Paragraph [0001] (“Die Erfindung betrifft eine wässrig-alkoholische Spraydesinfektionsmittelzubereitung zur Inaktivierung von behüllten und unbehüllten Viren auf unbelebten Oberflächen.”) The spray disinfectant contains from 30 to 70% of ethanol, an amine component and a terpene hydrocarbon against viruses. The methods of the present invention are intended for use on mammalian skin and do not involving dispensing terpene compounds on to skin.

The amine disclosed in EP0848907B1 is sold under the tradename LONZABAC® 12 from Lonza Group, Ltd (Basel, Switzerland). LONZABAC® 12 is laurylamine dipropylenediamine; it is not used as a neutralizing agent (as is the case in certain embodiments of the present invention that contain polyacrylate thickeners). LONZABAC® 12 is an amine-functionalized antimicrobial/disinfectant that is not intended for use on human skin; instead, it is intended for use on hard non-porous surfaces. See datasheet submitted in accompanying Information Disclosure Statement.

International Patent Application PCT/US2015/020145 published as WO 2015/138705 discloses a waterless and essentially alcohol-free sanitizer composition or aerosolizable hand sanitizing composition containing a quaternary ammonium salt (benzalkonium chloride) to kill germs and bacteria, a volatile carrier such as cyclomethicone, additional moisturizers, and optionally fragrance or other additives. The methods of the present invention do not involve dispensing silicone compounds on to skin.

US Pre-Grant Patent Application Publication (“PG Pub”) 2007/0281999 entitled “Alcohol-Containing Antimicrobial Compositions Having Improved Efficacy” discloses antimicrobial compositions having “rapid antibacterial effectiveness”, as well as “rapid and persistent antiviral effectiveness”, which are comprised of (i) a disinfecting alcohol (defined as a water-soluble alcohol having one to six carbon atoms, including ethanol, propanol, and isopropyl alcohol), (ii) water, and (iii) a blend containing a C₁₂ to C₂₂ alcohol and an ethoxylated C₁₂ to C₂₂ alcohol (e.g., cetearyl alcohol+cetereth-20; cetearyl alcohol+steareth-20+steareth-10). Paragraph [0308] of PG Pub 2007/0281999 broadly teaches that the disclosed compositions can be “applied to target animate or inanimate surface in several ways including spraying, misting, rolling, and foaming the composition onto the surface, or immersing the surface in the composition” The same paragraph generally teaches that application can be combined with physical agitation, such as spraying with pressure, rubbing, or brushing. This publication does not teach continuous or constant spraying. The methods of the present invention do not dispense ethoxylated C₁₂ to C₂₂ alcohols on to skin.

Aurena Laboratories (Karlstad, Sweeden) issued a press release on Feb. 26, 2020 describing an “alcohol-based disinfection spray for hands and body to reduce the risk of virus infections.” The product sheet from Aurena describes this disinfection spray as “contain[ing] only two ingredients, phospholipids and denatured alcohol”. See, https://www.aurenalabs.com/df_media/W1siZiIsIjIwMjAvMDMvMDEvMjMvNDgvMzAvO DdiMjAxNGQtNGVIOC00MDUxLTImNTEtNGNiNmNjM2Y2NzAwL1Byb2R1Y3RTaGVId HNTa2IuRGIzaW5mZWN0aW9uLnBkZiJdXQ/ProductSheetsSkinDisinfection.pdf?sha=0ea00ccb901ffb71 (accessed Apr. 2, 2020). The phospholipids in the Aurena product are trademarked under the AKVANO® brand by Lipidor, AB (Solna, Sweden). Lipidor AB describes AKVANO® technology as “based on water-free lipid formulations”.

Two bag-on-valve sprayable alcohol sanitizer with 62% ethanol as the active pharmaceutical ingredient have been sold under the brand names FACE VALUES® Hand Sanitizer Spray from Harmon Stores, Inc. and PERSANI™ Instant Hand Sanitizer. Both sprays contain, as inactive ingredients, Aloe and Vitamin E (or a Vitamin E derivative, tocopheryl acetate). Neither of these products contains an inert gas.

Importantly, methods of the present invention involve dispensing a hydroalcoholic mixture, which contain water at a concentration of at least 20% (vol/vol). Unlike the Aurena product, the methods of the present invention do not dispense fats or oils (or silicones) on skin.

Phospholipids—like soy lecithin used in the Aurena Skin Disinfectant Spray—have very limited solubility in water or alcohol. Assuming the Aurena product has a phospholipid content of at most 2% (approaching the solubility limit), the Aurena product would contain least 98% alcohol. Such a formulation would be less effective than a sanitizer that contains water. See, online article by Drs. Nina Gold and Usha Avva, physicians at Hackensack University Medical Center https://www.ncbi.nlm.nih.gov/books/NBK513254 (updated Feb. 6, 2020; accessed on Apr. 2, 2020)[The highest antimicrobial efficacy can be achieved with ethanol or isopropanol, respectively, at concentrations of 60-85% (vol/vol) and 60-80% (vol/vol)].

Hydrocarbon inhalation can exacerbate respiratory symptoms, and can cause inflammation, a condition that is prevalent in patients suffering from novel coronavirus 2019 (COVID-19). See, Brown KW and T J Armstrong, “Hydrocarbon Inhalation” https://www.ncbi.nlm.nih.gov/books/NBK470289/ (accessed on Apr. 10, 2020)(hydrocarbon exposure can trigger pneumonitis, a condition that can result in destruction of alveolar and capillary membranes as well as alteration of surfactant function and production, and, in certain cases, Acute Respiratory Distress Syndrome (ARDS) due to increased vascular permeability and edema).

With the current COVID-19 pandemic, there is a need for low cost, continuous spray, alcohol-based hand sanitizers. There is a need for providing a cost-effective tool to first responders and health care providers that rapidly and safely disinfects large areas of skin that may be contaminated with COVID-19. These needs are met by the methods of the present invention.

### BRIEF DESCRIPTION OF THE SEVERAL VIEWS OF THE DRAWINGS

FIG. 1 is a schematic cross-section of a valve assembly that is crimped to the end curl of the top-most cylindrical wall of a pressurized container body used in practicing the methods of the present invention.

FIG. 2 summarizes the different transfer broths and plating media used in testing certain embodiments of the present invention in accordance with method ASTM E2315-03(2008).

### SUMMARY OF INVENTION

A method for topical disinfection of mammalian skin comprising the steps of: (a) filling a container body with: (i) a lower alcohol selected from the group of C₁-C₆ alcohols or a mixture thereof at a concentration of from 24.00% to 45.00% vol/vol, based on the fill volume of the container, (ii) water at a concentration of from 8.00% to 12.00% vol/vol based on the fill volume of the container, (iii) a hydrophilic humectant at a concentration of from 0.04% to 4.25% vol/vol based on the fill volume of the container; (b) attaching and sealing a valve assembly to the container body; (c) filling the container body sealed with a valve assembly under a pressure of from 80 psi to 120 psi with a gaseous mixture of an inert gas, preferably nitrogen, (d) attaching an actuator to the valve assembly; and (e) depressing the actuator and dispensing a mixture of the lower alcohol, water, and hydrophilic humectant onto mammalian skin. 15 seconds after dispensing, the method achieves at least a 3−log₁₀ (99.9%) reduction in the concentration Gram-negative and Gram-positive microorganisms in accordance with method ASTM E2315-03(2008). Methods of the present invention can reduce levels of envelope viruses on human skin, including COVID-19.

### DETAILED DESCRIPTION OF INVENTION

The use of singular determiner (“a”, “an”, “the”) at the beginning of a noun phrase is to be understood to include plurals, unless the context dictates otherwise. For example, a reference to “an inert gas” includes more than one such gas.

“One or more” means at least one and thus includes individual components as well as mixtures/combinations.

Numbers used in describing quantities of ingredients and/or reaction conditions are to be understood as being modified in all instances by the term “about.”

Unless otherwise indicated, percentages, parts and ratios are to be understood as expressed on a volume/volume basis with reference to the total fill volume of a container that is pressurized to a pressure ranging from 70 psi to 130 psi.

Numerical ranges are meant to include numbers within the recited range, and combinations of subranges between, the given ranges. For example, a range from 1-5, includes 1, 2, 3, 4 and 5, as well as subranges such as 2-5, 3-5, 2-3, 2-4, 1-4, etc.

“At least one” means one or more, and also includes individual components as well as mixtures/combinations.

A “container body” is a vessel made of steel or aluminum, with a cylindrical wall, that is preferably straight. The bottom-most portion of the cylindrical wall is attached to an inwardly-curved (concave) bottom. The top-most portion of the cylindrical wall is inwardly-tapered to a curled end, which is attached to (crimped) to a valve assembly, as further described below. In preferred embodiments, the inner wall surface is coated with a corrosion-resistant polymeric coating that does not react with the materials that are filled, sealed, pressurized, and ultimately dispensed.

By “lower alcohol” is meant a C₁₋₆ alcohol. Non-limiting examples of C₁₋₆ alcohols include methanol, ethanol, propanol, isopropanol, butanol, isobutanol, tert-butanol, and mixtures of the foregoing.

By “coronavirus” is meant a family (Coronaviridae) of single-stranded RNA viruses that have a lipid envelope studded with club-shaped projections, infect birds and many mammals including humans, and include the causative agents of Middle East respiratory syndrome (MERS), severe acute respiratory syndrome (SARS), and COVID-19.

Preferably, the lower alcohol is ethanol, isopropanol, and/or n-propanol.

In certain preferred embodiments, the lower alcohol (or mixture of lower alcohols) is/are combined with a denaturant listed in 27 C.F.R. § 21.151.

In practicing the methods of the present invention, a container is filled with (i) a lower alcohol at a concentration of 24.00% to 45.00%; (ii) water at a concentration of from 8.00% to 12.00%; (iii) a hydrophilic humectant at a concentration of from 0.04% to 3.00%; and (iv) a gaseous mixture comprised of one or a mixture of inert gases at a concentration of from about 39.00% to about 59.00%.

The hydrophilic humectant is preferably selected from the group consisting of: glycerine; propane diols (e.g., methyl propane diol); propylene glycols (e.g., dipropylene glycol, triethylene glycol); short-chain polyethylene glycols (<200 mol wt); butylene glycols; pentylene glycols; hexylene glycols (e.g., hexane triols (1,2,6-hexanetriol); sorbitol, urea compounds; sodium pyrrolidone carboxylic acid; chitosan; and C₆₋₁₀ alkane diols (e.g, 1,2-hexanediol, 1,2-octanediol (aka caprylyl glycol), 1,9-nonanediol, 1,2-decanediol, 1,10-decanediol, or mixtures thereof).

Preferably, the hydrophilic humectant is selected from the group of glycerine and lower molecular weight polyols having from three to six carbon atoms.

Preferred C₃₋₆ polyols are selected from the group consisting of 1,3-propanediol, propylene glycol (1,2-dihydroxypropane), pentylene glycol (1,2-dihydroxpentane), and hexylene glycol (2-methyl-2,4-dihydroxpentane).

30 seconds after practicing the methods of the present invention—namely, dispensing the contents of the container onto mammalian skin—at least a 3−log₁₀ (99.9%) reduction in the viability of the following Gram-negative microorganisms is achieved:

(i) Haemophilus influenzae; (ii) Bacteroides fragilis; (iii) an Enterobacter species;

(iv) Burkholderia cepacia; (v) Escherichia coli; (vi) Klebsiella pneumoniae;

(vii) Pseudomonas aeruginosa; (viii) Serratia marcescens; (ix) Shigella sonnei; and

(x) Campylobacter jejuni; and (xi) Salmonella enterica.

After practicing methods of the present invention for 30 seconds, at least a 3−log₁₀ (99.9%) reduction in the viability of the following Gram-positive microorganisms is achieved: (i) Enterococcus faecalis; (ii) Staphylococcus aureus; (iii) Streptococcus pyogenes; (iv) Listeria monocytogenes; and (v) Streptococcus pneumoniae. Reduction in microorganism viability is determined in accordance with method ASTM E2315-03(2008).

Reduction in microorganism viability is determined in accordance with method ASTM E2315-03(2008).

The container is pressurized to 70 psi to 130 psi, preferably 80 psi to 120 psi, and most preferably from 90 psi to 110 psi.

The pressurized contents are dispensed onto mammalian skin by depressing an actuator on a valve assembly as described below.

30 seconds after dispensing, practicing the method of the present invention achieves at least a 4−log₁₀ (99.99%) reduction, more preferably at least a 5−log₁₀ (99.999%) reduction, and even more preferably a 6−log₁₀ (99.9999%) reduction in the concentration of the above-listed microorganisms, as measured by method ASTM E2315-03(2008).

15 seconds after dispensing, practicing the method of the present invention achieves at least a 4−log₁₀ (99.99%) reduction, more preferably at least a 5−log₁₀ (99.999%) reduction, and even more preferably a 6−log₁₀ (99.9999%) reduction in the concentration of the above-listed microorganisms, as measured by method ASTM E2315-03(2008).

In certain preferred embodiments the container is filled with a polymeric thickener. The polymeric thickener can be anionic, cationic, or non-ionic.

In certain preferred embodiments, the polymeric thickener is cationic and is synthesized from ethylenically unsaturated monomers of acrylates, acrylamides, vinyl lactams, vinyl acetates, methyl vinyl ethers, styrene, acrylonitrile, and vinyl pyrrolidone.

The polymeric thickener may be, and preferably is a homopolymer, for example the homopolymer of polyacrylic acid. One particularly preferred polymeric thickener is a homopolymer of Acrylic Acid crosslinked with an allyl ether of Pentaerythritol, an allyl ether of Sucrose, or an allyl ether of propylene commercially available as CARBOPOL® 980 (Lubrizol Advanced Materials, Inc., Cleveland, Ohio).

The polymeric thickener may be a crosspolymer of polyacrylic acid, for example Acrylates/C₁₀₋₃₀ Alkyl Acrylate Crosspolymer is a copolymer of C₁₀₋₃₀ Alkyl Acrylate and one or more monomers of Acrylic Acid, Methacrylic Acid or one of their esters crosslinked with an allyl ether of Sucrose or an allyl ether of Pentaerythritol, commercially available as CARBOPOL® 1382 (Lubrizol Advanced Materials, Inc., Cleveland, Ohio).

Another preferred group of polymeric thickeners is acrylate crosspolymers (e.g., acrylates/C₁₀₋₃₀ allyl acrylate, sold under the tradename Ultrez©)

Copolymers of acrylic acid and maleic anhydride; alkali swellable acrylates (e.g., Acusol); copolymers of polyvinyl pyrrolidone; and taurates (e.g., Aristoflex) are also suitable polymeric thickeners for inclusion in the continuous spray, hydroalcoholic, antiseptic sanitizer used to practice the methods of the present invention.

In certain embodiments in which the container is filled with a polymeric thickener, the container is also filled with neutralizing agent, which is preferably hindered base selected from hydroxides and amines.

Hindered bases that can serve as neutralizing agent in these embodiments, may be selected from the group consisting of: sodium hydroxide, ammonium hydroxide, potassium hydroxide; arginine; propoxylated ethylene diamines (e.g., tetrahydroxypropyl ethylenediamine, available under the tradename QUADROL® from BASF Corporation, Florham Park, N.J.); triethanolamine; tromethamine; triisopropanolamine; triisopropyl amine; 2-amino-2-methyl-1-propanol, 2-amino-2-methyl-1,3-propanediol, Tris (hydroxymethyl) aminomethane, and 2-dimethylamino-2-methyl-1-propanol (all available from Angus Chemical Company, Buffalo Grove, Ill.).

Preferred hindered bases are triethanolamine, 2-amino-2-methyl-1-propanol and tetrahydroxypropyl ethylenediamine.

In embodiments in which the container is filled with a polymeric thickener and a neutralizing agent, these ingredients are preferably present in a combined concentration of from 0.0125% to about 0.20%.

The container may be, and in certain preferred embodiments, is filled with an emollient ester selected from the group consisting of diesters of dibasic acids, triesters of citric acid, diesters of diols, triesters of triols, and combinations thereof.

Preferred emollient esters include isopropyl myristate, cetyl myristate, cetyl myristoleate, cetyl esters, diisopropyl sebacate, and diisopropyl adipate and 2-ethylhexyl palmitate, trimethylolpropane, neopentyl glycol

The container may be, and in certain preferred embodiments, is filled with one or more of (a) an anti-inflammatory botanical or marine extract and/or (b) a vitamin or a vitamin derivative and/or (c) an antimicrobial agent or preservative and/or (d) a fragrance and/or essential oil.

Preferred, but non-limiting examples of these anti-inflammatory ingredients include: avenanthramides (oat kernel extracts), tetracyclic and pentacyclic triterpene acids from plants in the genus Boswellia, salicylates (including Willow-bark extract), Aloe barbadensis, Arnica montana extract, Calendula officinalis, Hamamelis, Chamomile extract, Licorice extract (Dipotassium glycyrrhizinate, Stearyl glycyrrhetinate), alpine lichen, and algae extract.

One non-limiting, but preferred, vitamin derivative is Tocopheryl acetate.

The antimicrobial agent or preservative, when present, is not a paraben, formaldehyde or a formaldehyde donor, triclosan, methylchloroisothiazolinone, a carbamate, or an organotin.

In preferred embodiments, the combination of materials that are ultimately dispensed (under pressure) on to mammalian skin do not contain the following: fats; oils; silicones; surfactants; phthalates; para-aminobenzoic acid or other compounds listed on California's Safe Drinking Water and Toxic Enforcement Act of 1986, commonly known as “Proposition 65.”

The methods of the present invention are not practiced using a bag-on-valve dispenser and do not use hydrocarbon propellants, which can be irritating to the respiratory system. In addition, because they reduce the flash point of the mixture inside the pressurized container, inert gases are preferred over hydrocarbons.

The ratio of (i) lower alcohol, water and hydrophilic humectant, optionally with polymeric thickener and neutralizing agent) to (ii) inert gas mixture is from 7:13 to 13:7, and is preferably from 2:3 to 3:2.

Inert gases that can be used to fill a container in practicing the methods of the present invention include, but are not limited to, nitrogen, argon, carbon dioxide, nitrous oxide, a noble gas (listed in Group 18 on Periodic Table of Elements). Of these inert gases, nitrogen and argon are preferred; and nitrogen is most preferred.

After filling the container with at least one inert gas, the container may contain from about 3% to about 7% of compressed air, which, in turn, is comprised of about 21% oxygen and about 1% argon. The sealed container body may contain a “de minimis amount of other gases”, by which is meant less than 0.15% oxygen and argon.

Containers used in the methods of the present invention dispense a variety of spray patterns, where the spray pattern is determined by a combination of pressure, actuator selection, and other mechanical factors.

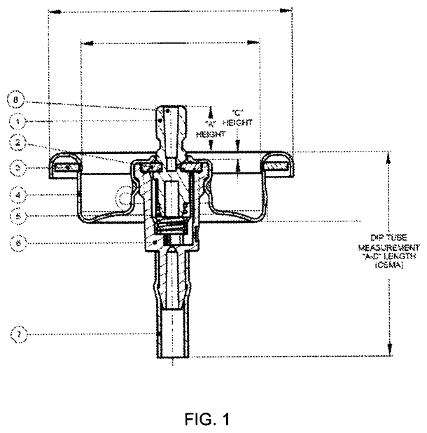

FIG. 1 is a cross section view of a valve assembly that is attached to a container used in methods of the present invention.

A valve stem assembly comprised of a valve stem [1], a stem gasket [3], a spring [5], and a dip tube [7]. The valve stem assembly controls delivery of the continuous spray hydroalcoholic antiseptic sanitizer.

A housing (also referred to as a container body) [6] encloses valve stem assembly.

A mounting cup gasket [2] forms a seal between the valve stem assembly and the container body.

A stem gasket [3] covers a stem orifice, and functions as an “on/off” switch.

A mounting cup [4] connects (seats) the valve assembly on the container body. A spring [5] closes the valve and preferably is made of stainless steel.

Pressure on an actuator [8] depresses the valve stem, interrupts the sealing action of the stem gasket and exposes a stem orifice to pressurized flow of continuous spray, hydroalcoholic, antiseptic sanitizer in the container, thereby opening the valve. When the actuator is released, the spring returns the stem orifice to a sealed position, closing the valve stem.

Valve piston dispensers are known in the art.

A user primes an actuator, causing a piston to compress a spring. Upward air pressure draws a ball from a resting position. Aerosolizable product is drawn into a dip tube and into a valve chamber.

When the user releases the actuator, the spring returns the piston to its starting (non-compressed) position, and the ball to its resting position. In the resting position, the ball seals the chamber, and prevents product from flowing backward. After priming, when a user depresses the actuator, product that is in the valve chamber is drawn through the actuator and dispensed.

Different types of actuators known in the art may be used to dispense a desired spray pattern.

The pressurized container may, and in certain embodiments, does contain an insert.

When present, the insert preferably has an orifice diameter of from about 0.007 inches to about 0.06 inches.

The actuator may, and preferably does, have a locking mechanism to prevent accidental discharge. One preferred locking mechanism is the Moritz twist-to-lock actuator from Aptar Group (Crystal Lake, Ill.).

A button actuator with an overcap may also be used.

The gasket(s) can be made from a wide variety of polymeric materials known to the skilled artisan, preferably a rubber.

Non-limiting examples of rubbers include, but not limited to: acrylonitrile butadiene unsaturated copolymers of 2-propenenitrile and various butadiene monomers (e.g., 1,2-butadiene and 1,3-butadiene); butyl rubber—a copolymer of isobutylene with isoprene; neoprene rubber—produced by polymerization of chloroprene; VITON®—a fluoroelastomer as defined in ASTM D1418; polyethylene (PE), including PE foam; and laminated materials.

In certain embodiments, the container body is made of steel, and a laminate gasket is used.

In certain embodiments, the pump/valve assembly is crimped onto the neck of the container body.

The dip tube is seated on or slightly above the bottom of the container and is preferably blunt, angled or notch cut.

The spray pattern is preferably cone-shaped.

The spray is preferably dispensed as a fine mist with a flood rate of from about 0.5 gram/second to 2.0 grams/second.

### EXAMPLES

The invention is further defined by reference to the container illustrated in FIG. 1 that is filled in accordance with Examples 1 and 2. These examples are representative, and should not be construed to limit the scope of the invention.

### Example 1

A container body is filled with a hydroalcoholic solution comprised of (i) ethanol and (ii) a mixture of water and glycerine, at a ratio of 7:3 on a volume/volume basis. The water-glycerine mixture is 94.667% water and 5.333% glycerine. The hydroalcoholic solution fills 60% of the container body. A valve assembly comprised of a Moritz actuator (Aptar Group, Crystal Lake, Ill.) with a 0.012 insert is attached (crimped) on the container body and is filled under a pressure of 97 psi with nitrogen, such that nitrogen is present at concentration of about 39.00% vol/vol, based on the fill volume of the container. An actuator is attached to the valve assembly, which is covered with an overcap.

### Example 2

A container body is filled with a hydroalcoholic solution comprised of (i) ethanol and (ii) a mixture of water and glycerine, at a ratio of 3:1 on a volume/volume basis. The water-glycerine mixture is 94.667% water and 5.333% glycerine. The container body is sealed with a valve assembly and filled with nitrogen gas in the same manner as Example 1.

The overcap is removed, and the actuator is pressed, dispensing the pressurized contents on to a human hand. As dispensed, the pressurized contents form a continuous spray, hydroalcoholic, antiseptic sanitizer, and deposit on the skin in a circular spray pattern with the following dimensions:

| Distance | Diameter | Total coverage area |
|---|---|---|
| 2 in. | 2.75 in. | 5.94 sq. in. |
| 3 in. | 3.50 in. | 9.62 sq. in. |
| 6 in. | 5.00 in. | 19.60 sq. in. |

The flood rate of the continuous spray, hydroalcoholic, antiseptic sanitizer from the pressurized container is about 1 gram/second.

### Example 3

In accordance with procedures recommended by the American Society of Microbiology [see, Balows, A and W J Hausler eds., “Manual of Clinical Microbiology,” (5th ed., 1991)] and a test method promulgated by the American Society of Testing and Materials [ASTM E2315-03(2008) Standard Guide for Assessment of Antimicrobial Activity Using a Time-Kill Procedure], time-dependent bactericidal activity (also known in the art as antimicrobial efficacy) is evaluated against the following clinically relevant microorganisms—26 repository strains of Gram-negative and Gram-positive bacteria obtained from American Type Culture Collection (“ATCC”) in Manassas, Va.:
(i) Acinetobacter baumannii, ATCC 19606
(ii) Bacteroides fragilis, ATCC 25285
(iii) Burkholderia cepacia, ATCC 25416
(iv) Burkholderia cepacia, ATCC 25608
(v) Campylobacter jejuni, ATCC 33291
(vi) Campylobacter jejuni, ATCC 49943
(vii) Candida albicans, ATCC 10231
(viii) Candida glabrata, ATCC 26512
(ix) Enterobacter aerogenes, ATCC 13048
(x) Enterococcus faecalis, ATCC 19433
(xi) Enterococcus faecalis, ATCC 29212
(xii) Enterococcus faecium, ATCC 19434
(xiii) Escherichia coli, ATCC 11229
(xiv) Escherichia coli, ATCC 11775
(xv) Escherichia coli, ATCC 25922
(xvi) Haemophilus influenzae, ATCC 19418
(xvii) Klebsiella pneumoniae, ATCC 13883
(xviii) Klebsiella pneumoniae, ATCC 27736
(xix) Klebsiella pneumoniae, ATCC 4352
(xx) Listeria monocytogenes, ATCC 19115
(xxi) Listeria monocytogenes, ATCC 7644
(xxii) Micrococcus yunnanensis, ATCC 7468
(xxiii) Proteus mirabilis, ATCC 7002
(xxiv) Pseudomonas aeruginosa, ATCC 15442
(xxv) Pseudomonas aeruginosa, ATCC 27853
(xxvi) Salmonella enterica serovar enteritidis, ATCC 13076
(xxvii) Salmonella enterica serovar typhimurium, ATCC 14028
(xxviii) Serratia marcescens, ATCC 8100
(xxix) Serratia marcescens, ATCC 14756
(xxx) Shigella sonnei, ATCC 25931
(xxxi) Shigella sonnei, ATCC 9290
(xxxii) Staphylococcus aureus MRSA, ATCC 33591
(xxxiii) Staphylococcus aureus MRSA, ATCC 33592
(xxxiv) Staphylococcus aureus, ATCC 29213
(xxxv) Staphylococcus aureus, ATCC 6538
(xxxvi) Staphylococcus epidermidis, ATCC 12228
(xxxvii) Staphylococcus haemolyticus, ATCC 29970
(xxxviii) Staphylococcus hominis, ATCC 27844
(xxxix) Staphylococcus saprophyticus, ATCC 15305
(xl) Streptococcus pneumoniae, ATCC 6303
(xli) Streptococcus pneumoniae, ATCC 33400
(xlii) Streptococcus pneumoniae, ATCC 49619
(xliii) Streptococcus pyogenes, ATCC 14289
(xliv) Streptococcus pyogenes, ATCC 19615

Each of the above-listed microorganisms are transferred from ATCC stock cultures into appropriate broth media and incubated for 18-24 hours at 36±1° C.

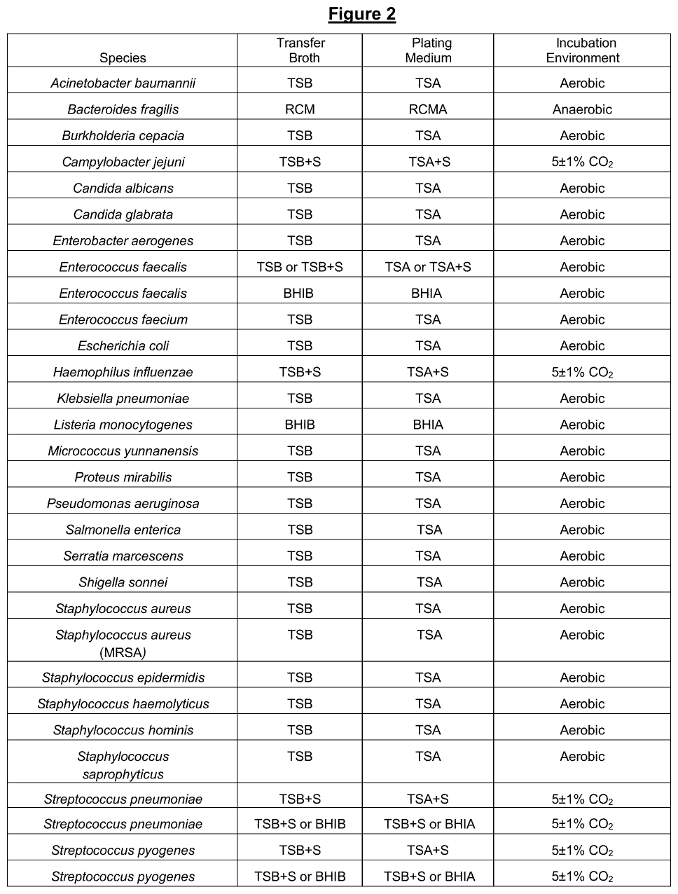

FIG. 2 summarizes the different transfer broths used: Tryptic Soy Broth (TSB); Tryptic Soy Broth containing 5% Defibrinated Sheep's Blood (TSB+S); Reinforced Clostridial Medium (RCM); and Brain Heart Infusion Broth (BHIB).

Using Butterfield's Phosphate Buffered Dilution Water (PBDW), an inoculum is prepared for each challenge microorganism at a concentration of 1×10⁸ CFU/ml.

Test Groups: Three replicates of the continuous spray, hydroalcoholic, antiseptic sanitizers of each of Examples 1 and 2 are prepared by adding 0.10 ml of inoculum of each challenge microorganism to 9.9 ml of test product in a test tube and mixing. At 15 seconds and 30 seconds, a 1 ml sample is removed and added to a tube containing 9 ml of neutralizer (0.3% Lecithin, 1.0% Tween® 80, and 1.0% Tamol®).

Control Groups: Six replicates (three for Example 1 and three for Example 2) are prepared by adding 0.10 ml of inoculum of each challenge microorganism to 9.9 ml of PBDW in a test tube and mixing. At 15 seconds and 30 seconds, a 1 ml sample is removed and added to a tube containing 9 ml of neutralizer (0.3% Lecithin, 1.0% Tween® 80, and 1.0% Tamol®).

For the Test Groups and the Control Groups, serial ten-fold dilutions are performed with PBDW. Duplicate aliquots from selected dilutions are prepared and incubated at 36±1° C. for 48±2 hours on pour plates (1-mL aliquots) or spread plates (0.1-mL aliquots). (Agar medium and incubation conditions for each microorganism are indicated in FIG. 1.)

FIG. 2 summarizes the different plating media used: Tryptic Soy Agar (TSA); Tryptic Soy Agar containing 5% Defibrinated Sheep's Blood (TSA+S); Reinforced Clostridial Medium Agar (RCMA); Brain Heart Infusion Agar (BHIA). TSA+S is used for spread plating. RCMA or BHIA is used for pour plating.

Plates are removed from the incubator, colonies are counted, and concentration (CFU/ml) enumerated at each of the two contact times.

Log₁₀ reductions are calculated by subtracting the average log₁₀ recovery of the evaluated test product (test or control) at each time point (15 seconds and 30 seconds).

With a contact time of 15 seconds (after application), each of the continuous spray, hydroalcoholic, antiseptic sanitizer formulations of Examples 1 and 2 achieve a 6−log₁₀ reduction in the level of each of the 26 challenge microorganisms.

While the illustrative embodiments of the invention have been described with particularity, it will be understood that various modifications will be apparent to (and can be readily made by) those skilled in the art without departing from the spirit and scope of the invention.

Accordingly, it is not intended that the scope of the claims be limited to the examples and descriptions set forth above. The claims are to be construed as encompassing all of the features of patentable novelty in the present invention, including all features which would be treated as equivalents by persons having ordinary skill in the art of formulating personal care products in the form of sprays intended for direct application to human skin.

1. A method for topical disinfection of mammalian skin comprising the steps of
(a) filling a container body with
(i) a lower alcohol selected from the group of C₁-C₆ alcohols or a mixture thereof at a concentration of from 24.00% to 45.00% vol/vol, based on the fill volume of the container,
(ii) water at a concentration of from 8.00% to 12.00% vol/vol, based on the fill volume of the container
(iii) a hydrophilic humectant at a concentration of from 0.04% to 3.00% vol/vol based on the fill volume of the container
(b) attaching and sealing a valve assembly to the container body
(c) filling the container body sealed with a valve assembly under a pressure of from 80 psi to 120 psi with an inert gas at a concentration of from about 39.00% to about 59.00% vol/vol, based on the fill volume of the container,
(d) attaching an actuator to the valve assembly; and
(e) depressing the actuator and dispensing a mixture of the lower alcohol, water, and hydrophilic humectant onto mammalian skin
wherein 15 seconds after dispensing the method achieves
(xx) at least a 3−log₁₀ (99.9%) reduction in the concentration of the following Gram-negative microorganisms in accordance with method ASTM E2315-03(2008): (i) Haemophilus influenzae; (ii) Bacteroides fragilis; (iii) an Enterobacter species; (iv) Burkholderia cepacia; (v) Escherichia coli; (vi) Klebsiella pneumoniae; (vii) Pseudomonas aeruginosa; (viii) Serratia marcescens; (ix) Shigella sonnei; and (x) Campylobacter jejuni; and (xi) Salmonella enterica.
(yy) at least a 3−log 10 (99.9%) reduction in the concentration of the following Gram-positive microorganisms in accordance with method ASTM E2315-03(2008): (i) Enterococcus faecalis; (ii) Staphylococcus aureus; (iii) Streptococcus pyogenes; (iv) Listeria monocytogenes; and (v) Streptococcus pneumoniae.

2. The method of claim 1 wherein the lower alcohol is ethanol.

3. The method of claim 2 wherein the lower alcohol is present at a concentration of from 42% vol/vol to 45% vol/vol, and further wherein within 15 seconds after dispensing, the method achieves at least a 4−log₁₀ (99.99%) reduction in the concentration of the following microorganisms, as measured in accordance with method ASTM E2315-03(2008): (i) Haemophilus influenzae; (ii) Bacteroides fragilis; (iii) an Enterobacter species; (iv) Burkholderia cepacia; (v) Escherichia coli; (vi) Klebsiella pneumoniae; (vii) Pseudomonas aeruginosa; (viii) Serratia marcescens; (ix) Enterococcus faecalis; (x) Staphylococcus aureus; (xi) Streptococcus pyogenes; (xii) Listeria monocytogenes; and (xiii) Streptococcus pneumoniae.

4. The method of claim 3 wherein within 15 seconds after dispensing the method achieves at least a 5−log₁₀ (99.999%) reduction in the concentration of the microorganisms, as measured in accordance with method ASTM E2315-03(2008).

5. The method of claim 4 wherein the inert gas is nitrogen.

6. The method of claim 5 wherein the hydrophilic humectant is glycerine.

7. The method of claim 6 wherein the container is pressurized to 90 psi to 110 psi.

8. The method of claim 7, wherein step (a) further comprises filling the container with (iv) a hydrophilic polymeric thickener and (v) a neutralizing agent at a combined concentration of from 0.0125% to 0.20% vol/vol, based on the fill volume of the container.

9. The method of claim 8 wherein the hydrophilic polymeric thickener is a homopolymer of polyacrylic acid and the neutralizing agent is a hindered amine.

10. The method of claim 1 wherein 30 seconds after dispensing, the method reduces the concentration of COVID-19 on skin surface.

11. The method of claim 10 wherein the level of COVID-19 is reduced by 3−log₁₀.
